(19) United States
(12) Patent Application Publication
(10) Pub. No.: US 2005/0171210 A1
(43) Pub. Date: Aug. 4, 2005

(54) Compositions and methods for treatment of cardiovascular disorders and diseases

(75) Inventors: Moussa B.H. Youdim, Haifa (IL); Ofer Binah, Nofit (IL); Zaid A. Abassi, Haifa (IL); Yaron Barac, Kiryat Motzkin (IL)

Correspondence Address: BROWDY AND NEIMARK, P.L.L.C.;624 NINTH STREET, NW, SUITE 300, WASHINGTON, DC, 20001-5303, US

(73) Assignees: Technion Research and Development Foundation Ltd., Haifa (IL); Rappaport Family Institute For Research In The Medical Sciences, Haifa (IL)

(21) Appl. No.: 10/952,367

(22) Filed: Sep. 29, 2004

Related U.S. Application Data
(60) Provisional application No. 60/524,616, filed on Nov. 25, 2003.
(60) Provisional application No. 60/570,496, filed on May 13, 2004.

(51) Int. Cl. A61K 31/135; A61K 31/205

(52) U.S. Cl. 514/657; 514/554

(57) ABSTRACT

Propargylamine, propargylamine derivatives including N-propargyl-1-aminoindan and analogs thereof, and pharmaceutically acceptable salts thereof, are useful for prevention or treatment of cardiovascular disorders and diseases.

### FIELD AND BACKGROUND OF THE INVENTION

The present invention relates to compositions and methods for the treatment of cardiovascular disorders and diseases and, more particularly, to propargylamine and derivatives thereof for use in said compositions and methods.

### Cardiovascular Disorders and Diseases

Cardiovascular disorders and diseases and their associated complications are a principal cause of disabilities and deaths of individuals in the United States and Western Europe. For example, in recent years more than 500,000 deaths have occurred annually in the United States alone as a result of coronary artery disease, and an additional 700,000 patients have been hospitalized for myocardial infarction.

Ischemic heart disease (IHD) is the most common, serious, chronic, life-threatening illness among the cardiovascular disorders and diseases. Ischemia, reduced myocardial perfusion, which causes lack of oxygen (hypoxia) as well as other metabolic changes, is the most common effect resulting from an inadequate blood flow through the coronary arteries, which are the blood suppliers of the heart. The most common cause of myocardial ischemia is the atherosclerotic disease of epicardial coronary arteries. The plaques consist of subintimal collections of fat, cells, and debris, which develop at irregular rates in different segments of the epicardial coronary tree, and lead eventually to segmental reductions in cross-sectional area (stenosis). When the coronary artery cross-section area is reduced by ˜75%, a full range of increases in flow to meet increased myocardial demand is not possible. When the luminal area is reduced by more than 80%, blood flow at rest may be reduced, and further minor decreases in the stenotic orifice can reduce coronary flow dramatically and cause myocardial ischemia and infarction. This situation impairs myocardial contractility during exercise, creating the chest angina. Critical stenosis of the coronaries can cause chest angina even at rest, implying that the myocardium is suffering from lack of perfusion. The most serious complication of ischemic heart disease is acute myocardial infarction (AMI), which is one of the most common diagnoses in hospitalized patients. AMI generally occurs when coronary blood flow decreases abruptly after a thrombotic occlusion of a coronary artery, previously narrowed by atherosclerotic plaque. Although the mortality rate after admission for AMI has declined by about 30% over the last two decades, approximately 1 of every 25 patients who survives the initial hospitalization dies in the first year after AMI. The first step is the dissection of the atherosclerotic plaque, which causes the exposure of the thrombogenic plaque core to the blood. Because of its high thrombogenicity, a thrombus consists mainly of fibrin and activated. thrombocyte is rapidly growing from the plaque core. Consequently, blood flow is seriously disturbed until there is no sufficient blood flow to the myocardium and an infarction begins due to lack of perfusion of oxygen.

There are various insults which cause the myocardial damage during myocardial ischemia and infarction. Lack of adequate perfusion to the heart tissue may result in: (i) lack of oxygen (hypoxia); (ii) growth factors and nutrients deprivation (e.g., IGF-1, insulin, glucose); (iii) acidosis (lactic acid production); (iv) hyperkalemia (due to environmental acidosis and cell damage); and/or (v) during ischemia, but mostly following reperfusion (resumption of the blood flow to the ischemic tissue), reactive oxygen species (ROS) such as H₂O₂, O₂⁻ OH⁻ are created by the ischemic cells and by damage to neighboring cells. Each and every of these insults has been shown to exert damage to cardiomyocytes both in vivo and in vitro.

Two main cellular death types occur in nature: necrosis and apoptosis. While necrosis is a random process, often initiated by hostile environmental stimuli, apoptosis is a programmed cell death in which distinct intracellular signaling pathways are activated. Apoptosis is a fundamental physiological and pathologic mechanism that allows elimination of no-longer useful cells during embryogenesis, or of aged or damaged cells during life. Unlike necrosis, which involves large number of cells, apoptosis usually affects small number of cells without inflammation. In apoptosis, the nuclear DNA is “digested” into small fragments by special DNAses, while cytoskeletal and myofibrillar proteins are degraded by specialized proteases as well. At the final stages, the cell dissolves into a characteristic membrane bound vesicles (apoptotic bodies), which are quickly phagocyted by phagocytic neighboring cells.

Cells can undergo apoptosis caused by intrinsic stimuli, e.g. hypoxia, ROS, chemotherapies, or by extrinsic stimuli, mainly referred to activation of death receptors such as TNF-α and Fas.

Fas is a ubiquitous cell-surface receptor involved in apoptosis initiation. Fas belongs to the TNF/NGF superfamily, and is activated by Fas Ligand (FasL), which may cause apoptosis in Fas-bearing cells (Berke, 1997). It appears that while healthy cardiomyocytes are resistant to Fas-mediated apoptosis, during cardiac pathologies cardiomyocytes become sensitive to Fas-mediated apoptosis. Recent studies suggest that in several important heart diseases such as myocarditis, hypertrophy, ischemia, ischemia/reperfusion and heart failure, Fas activation results in apoptotic as well as in non-apoptotic effects, both contributing to cardiac dysfunction (Haunstetter and Izumo, 1998; Binah, 2000). Recent studies have shown that Fas activation is involved not only in myocardial pathologies inflicted by immune effectors (CTLs) such as transplant rejection, myocarditis and the resulting dilated cardiomyopathy (Binah, 2000; Hershkowitz et al., 1987), but also in lymphocyte-independent diseases such as ischemia/reperfusion injuries (Fliss and Gattinger, 1996; Yaoita et al., 1998; Jeremias et al., 2000). In this regard, it was recently proposed that FasL can be cleaved by a metalloprotease to form soluble FasL (sFasL), which can cause apoptosis in susceptible cells. Therefore, sFasL, which may be secreted from the failing heart and is elevated in patients with advanced congestive heart failure (Yamaguchi et al., 1999), is a potential contributor to apoptosis in this wide-spread heart pathology.

Programmed cell death (apoptosis) is recognized, increasingly, as a contributing cause of cardiac myocyte loss with ischemia/reperfusion injury, myocardial infarction, and long-standing heart failure.

### Propargylamine and Propargylamine Derivatives

Several propargylamine derivatives have been shown to selectively inhibit monoamine oxidase (MAO)-B and/or MAO-A activity and, thus to be suitable for treatment of neurodegenerative diseases such as Parkinson's and Alzheimer's disease. In addition, these compounds have been further shown to protect against neurodegeneration by preventing apoptosis.

Rasagiline, R(+)-N-propargyl-1-aminoindan, a highly potent selective irreversible monoamine oxidase (MAO)-B inhibitor, has been shown to exhibit neuroprotective activity and antiapoptotic effects against a variety of insults in cell cultures and in vivo and has finished recently the phase III clinical trials for Parkinson's disease.

R(+)-N-propargyl-1-aminoindan and pharmaceutically acceptable salts thereof were first disclosed in U.S. Patents U.S. Pat. No. 5,387,612, U.S. Pat. No. 5,453,446, U.S. Pat. No. 5,457,133, U.S. Pat. No. 5,576,353, U.S. Pat. No. 5,668,181, U.S. Pat. No. 5,786,390, U.S. Pat. No. 5,891,923, and U.S. Pat. No. 6,630,514 as useful for the treatment of Parkinson's disease, memory disorders, dementia of the Alzheimer type, depression, and the hyperactive syndrome. The 4-fluoro-, 5-fluoro- and 6-fluoro-N-propargyl-1-aminoindan derivatives were disclosed in U.S. Pat. No. 5,486,541 for the same purposes.

U.S. Pat. No. 5,519,061, U.S. Pat. No. 5,532,415, U.S. Pat. No. 5,599,991, U.S. Pat. No. 5,744,500, U.S. Pat. No. 6,277,886, U.S. Pat. No. 6,316,504, US 133, U.S. Pat. No. 5,576,353, U.S. Pat. No. 5,668,181, U.S. Pat. No. 5,786,390, U.S. Pat. No. 5,891,923, and U.S. Pat. No. 6,630,514 disclose R(+)-N-propargyl-1-aminoindan and pharmaceutically acceptable salts thereof as useful for treatment of additional indications, namely, an affective illness, a neurological hypoxia or anoxia, neurodegenerative diseases, a neurotoxic injury, stroke, brain ischemia, a head trauma injury, a spinal trauma injury, schizophrenia, an attention deficit disorder, multiple sclerosis, and withdrawal symptoms.

U.S. Pat. No. 6,251,938 describes N-propargyl-phenylethylamine compounds, and U.S. Pat. No. 6,303,650, U.S. Pat. No. 6,462,222 and U.S. Pat. No. 6,538,025 describe N-propargyl-1-aminoindan and N-propargyl-1-aminotetralin compounds, said to be useful for treatment of depression, attention deficit disorder, attention deficit and hyperactivity disorder, Tourette's syndrome, Alzheimer's disease and other dementia such as senile dementia, dementia of the Parkinson's type, vascular dementia and Lewy body dementia.

The first compound found to selectively inhibit MAO-B was R-(−)-N-methyl-N-(prop-2-ynyl)-2-aminophenylpropane, also known as L-(−)-deprenyl, R-(−)-deprenyl, or selegiline. In addition to Parkinson's disease, other diseases and conditions for which selegiline is disclosed as being useful include: drug withdrawal (WO 92/21333, including withdrawal from psychostimulants, opiates, narcotics, and barbiturates); depression (U.S. Pat. No. 4,861,800); Alzheimer's disease and Parkinson's disease, particularly through the use of transdermal dosage forms, including ointments, creams and patches; macular degeneration (U.S. Pat. No. 5,242,950); age-dependent degeneracies, including renal function and cognitive function as evidenced by spatial learning ability (U.S. Pat. No. 5,151,449); pituitary-dependent Cushing's disease in humans and nonhumans (U.S. Pat. No. 5,192,808); immune system dysfunction in both humans (U.S. Pat. No. 5,387,615) and animals (U.S. Pat. No. 5,276,057); age-dependent weight loss in mammals (U.S. Pat. No. 5,225,446); schizophrenia (U.S. Pat. No. 5,151,419); and various neoplastic conditions including cancers, such as mammary and pituitary cancers. WO 92/17169 discloses the use of selegiline in the treatment of neuromuscular and neurodegenerative disease and in the treatment of CNS injury due to hypoxia, hypoglycemia, ischemic stroke or trauma. In addition, the biochemical effects of selegiline on neuronal cells have been extensively studied (e.g., see Tatton, et al., 1991 and 1993). U.S. Pat. No. 6,562,365 discloses the use of desmethylselegiline for selegiline-responsive diseases and conditions.

U.S. Pat. No. 5,169,868, U.S. Pat. No. 5,840,979 and U.S. Pat. No. 6,251,950 disclose aliphatic propargylamines as selective MAO-B inhibitors, neuroprotective and cellular rescue agents. The lead compound, (R)-N-(2-heptyl)methyl-propargylamine (R-2HMP), has been shown to be a potent MAO-B inhibitor and antiapoptotic agent (Durden et al., 2000).

Propargylamine was reported many years ago to be a mechanism-based inhibitor of the copper-containing bovine plasma amine oxidase (BPAO), though the potency was modest. U.S. Pat. No. 6,395,780 discloses propargylamine as a weak glycine-cleavage system inhibitor. U.S. Provisional Application No. 60/541,066 entitled “Use of propargylamine as neuroprotective agent”, filed 3 Feb. 2004, herewith incorporated by reference in its entirety as if fully disclosed herein, discloses that propargylamine exhibits neuroprotective and anti-apoptotic activities and can, therefore, be used for all known uses of rasagiline and similar drugs containing the propargylamine moiety.

### SUMMARY OF THE INVENTION

The present invention relates to a method for treatment of a subject susceptible to or suffering from a cardiovascular disorder or disease which comprises administering to the subject an amount of an agent selected from the group consisting of propargylamine, a propargylamine derivative and a pharmaceutically acceptable salt thereof, effective to treat the subject.

In one preferred embodiment of the invention, the agent is propargylamine or a pharmaceutically acceptable salt thereof. In another preferred embodiment, the agent is a propargylamine derivative, more preferably an N-propargyl-1-aminoindan (also known as rasagiline), an enantiomer thereof, an analog thereof, or a pharmaceutically acceptable salt thereof and, most preferably, it is rasagiline.

The methods and compositions of the invention are suitable for preventing and/or treating congestive heart failure, cardiac hypertrophy including both atrial and ventricular hypertrophy, myocardial infarction, myocardial ischemia, myocardial ischemia and reperfusion, or arrhythmias.

### BRIEF DESCRIPTION OF THE FIGURES

FIG. 1 depicts apoptosis induced in H9c2 heart cell line by means of recombinant Fas ligand (rFasL). The apoptotic cells detected by DAPI staining are marked by the arrows (right side).

FIG. 2 shows that rasagiline blocks Fas-mediated apoptosis in H9c2 cells.

FIG. 3 shows that rasagiline protects against serum starvation-induced apoptosis in H9c2 cells.

FIG. 4 shows that rasagiline protects against serum starvation-mediated but not H₂O₂- induced apoptosis in H9c2 cells. * compared to control. ** compared to serum starvation (p<0.05).

FIG. 5 shows that propargylamine blocks Fas-mediated hypertrophy in cultured neonatal rat ventricular myocytes. The top panel depicts representative atrial natriuretic peptide (ANP) mRNA blots in Control, rFasL, and in rFasL+propargylamine. The lower panel depicts the summary of three experiments. Hypertrophy was expressed as the ratio between ANP and actin. * P<0.05 vs. control.

### DETAILED DESCRIPTION OF THE INVENTION

The present invention relates to a method for treatment of a subject susceptible to or suffering from a cardiovascular disorder or disease which comprises administering to the subject an amount of an agent selected from the group consisting of propargylamine, a propargylamine derivative, or a pharmaceutically acceptable salt thereof, effective to treat the subject.

The present invention further relates to a method for treatment of a subject susceptible to or suffering from a cardiovascular disorder or disease which comprises administering to the subject an amount of an agent selected from the group consisting of propargylamine, a propargylamine derivative, or a pharmaceutically acceptable salt thereof, effective to protect ventricular muscle from apoptosis, particularly Fas-mediated apoptosis, wherein said cardiovascular disorder or disease is ischemia/reperfusion injury, myocardial infarction, and long-standing heart failure.

In one preferred embodiment, the active agent used in the present invention is propargylamine or a pharmaceutically acceptable salt thereof. The use of any physiologically acceptable salt of propargylamine is encompassed by the present invention such as the hydrochloride, hydrobromide, sulfate, mesylate, esylate, tosylate, sulfonate, phosphate, or carboxylate salt. In more preferred embodiments, propargylamine hydrochloride and propargylamine mesylate are used according to the invention.

In another preferred embodiment, the active agent used in the present invention is N-propargyl-1-aminoindan, either in its racemic form (described, for example, in U.S. Pat. No. 6,630,514) or as the R-enantiomer R(+)-N-propargyl-1-aminoindan (described, for example, in U.S. Pat. No. 5,387,612) or as the S-enantiomer S-(−)-N-propargyl-1-aminoindan (described, for example, in U.S. Pat. No. 6,277,886). In a more preferred embodiment of the invention, the active agent is rasagiline, the R(+)-N-propargyl-1-aminoindan.

In another preferred embodiment, the active agent is a pharmaceutically acceptable salt of N-propargyl-1-aminoindan or of an enantiomer thereof including, but not limited to, the mesylate, maleate, fumarate, tartrate, hydrochloride, hydrobromide, esylate, p-toluenesulfonate, benzoate, acetate, phosphate and sulfate salts. In preferred embodiments, the salt is a pharmaceutically acceptable salt of R(+)-N-propargyl-1-aminoindan such as, but not limited to, the mesylate salt (described, for example, in U.S. Pat. No. 5,532,415), the esylate and the sulfate salts (both described, for example, in U.S. Pat. No. 5,599,991), and the hydrochloride salt (described, for example, in U.S. Pat. No. 6,630,514) of R(+)-N-propargyl-1-aminoindan.

In a further embodiment, the active agent is an analog of N-propargyl-1-aminoindan, an enantiomer or a pharmaceutically acceptable salt thereof. In one embodiment, the analogs are the compounds described in U.S. Pat. No. 5,486,541 such as, but not limited to, the compounds 4-fluoro-N-propargyl-1-aminoindan, 5-fluoro-N-propargyl-1-aminoindan, 6-fluoro-N-propargyl-1-aminoindan, an enantiomer thereof and pharmaceutically acceptable addition salts thereof. In another embodiment, the analogs are the compounds described in U.S. Pat. No. 6,251,938 such as, but not limited to, the compounds (rac)-3-(N-methyl,N-propyl-carbamyloxy)-α-methyl-N′-propargyl phenethylamine HCl; (rac)-3-(N,N-dimethyl-carbamyloxy)-α-methyl-N′-methyl, N′-propargyl phenethylamine HCl; (rac)-3-(N-methyl,N-hexyl-carbamyloxy)-α-methyl-N′-methyl, N′-propargyl phenethylamine mesylate; (rac)-3-(N-methyl, N-cyclohexyl-carbamyloxy)-α-methyl-N′-methyl,N′-propargylphenethyl HCl; and (S)-3-(N-methyl, N-hexyl-carbamyloxy)-α-methyl-N′-methyl,N′-propargyl phenethylamine ethane-sulfonate. In a further embodiment, the analogs are the compounds described in U.S. Pat. No. 6,303,650 such as, but not limited to, the compounds (rac) 6-(N-methyl, N-ethyl-carbamyloxy)-N′-propargyl-1-aminoindan HCl; (rac) 6-(N,N-dimethyl, carbamyloxy)-N′-methyl-N′-propargyl-1-aminoindan HCl; (rac) 6-(N-methyl, N-ethyl-carbamyloxy-N′-propargyl-1-aminotetralin HCl; (rac) 6-(N,N-dimethyl-thiocarbamyloxy)-1-aminoindan HCl; (rac) 6-(N-propyl-carbamyloxy-N′-propargyl-1-aminoindan HCl; (rac) 5-chloro-6-(N-methyl, N-propyl-carbamyloxy)-N′-propargyl-1-aminoindan HCl; (S)-6-(N-methyl), N-propyl-carbamyloxy)-N′-propargyl-1-aminoindan HCl; and (R)-6-(N-methyl, N-ethyl-carbamyloxy)-N′-propargyl-1-aminoindan hemi-(L)-tartrate, and 6-(N-methyl, N-ethyl-carbamyloxy)-N′-methyl,N′-propargyl-1-aminoindan described in U.S. Pat. No. 6,462,222.

In a still further embodiment, the active agent is an aliphatic propargylamine described in U.S. Pat. No. 5,169,868, U.S. Pat. No. 5,840,979 and U.S. Pat. No. 6,251,950 such as, but not limited to, the compounds N-(1-heptyl)propargylamine; N-(1-octyl)propargylamine; N-(1-nonyl) propargylamine; N-(1-decyl)propargylamine; N-(1-undecyl)propargylamine: N-(1-dodecyl) propargylamine; R-N-(2-butyl) propargylamine; R-N-(2-pentyl) propargylamine; R-N-(2-hexyl)propargylamine; R-N-(2-heptyl)propargylamine; R-N-(2-octyl) propargylamine; R-N-(2-nonyl) propargylamine; R-N-(2-decyl) propargylamine, R-N-(2-undecyl)propargylamine; R-N-(2-dodecyl)propargylamine: N-(1-butyl)-N-methylpropargylamine; N-(2-butyl)-N-methylpropargylamine ; N-(2-pentyl)-N-methylpropargylamine; N-(1-pentyl)-N-methylpropargylamine; N-(2-hexyl)-N-methylpropargylamine; N-(2-heptyl)-N-methylpropargylamine; N-(2-decyl)-N-methylpropargylamine; N-(2-dodecyl)-N-methylpropargylamine; R(−)-N-(2-butyl)-N-methylpropargylamine; or a pharmaceutically acceptable salt thereof.

In yet another embodiment, the active agent is selegiline, desmethylselegiline or norprenyl, pargyline or chlorgyline.

In still another embodiment, the active agent is the compound (N-methyl-N-propargyl-10-aminomethyl-dibenzo[b,f]oxepin (known as CGP 3466, described in Zimmermann et al., 1999).

All the US patents and other publications mentioned hereinabove are hereby incorporated by reference in their entirety as if fully disclosed herein.

In another aspect, the present invention provides a pharmaceutical composition for prevention and/or treatment of a cardiovascular disorder or disease comprising a pharmaceutically acceptable carrier and an agent selected from the group consisting of propargylamine, a propargylamine derivative, or a pharmaceutically acceptable salt thereof as described above.

The pharmaceutical composition provided by the present invention may be in solid, semisolid or liquid form and may further include pharmaceutically acceptable fillers, carriers or diluents, and other inert ingredients and excipients. The composition can be administered by any suitable route, e.g. intravenously, orally, parenterally, rectally, or transdermally. The dosage will depend on the state of the patient and severity of the disease and will be determined as deemed appropriate by the practitioner.

In one embodiment, the pharmaceutically acceptable carrier is a solid and the pharmaceutical composition is in a suitable form for oral administration including tablets, compressed or coated pills, dragees, sachets, hard or soft gelatin capsules, and sublingual tablets. In a more preferred embodiment, the pharmaceutical composition is a tablet containing an amount of the active agent in the range of about 0.1-100 mg, preferably from about 1 mg to about 10 mg.

In another embodiment, the pharmaceutically acceptable carrier is a liquid and the pharmaceutical composition is an injectable solution. The amount of the active agent in the injectable solution is in the range of from about 0.1 mg/ml to about 100 mg/ml, more preferably 1 mg/ml to about 10 mg/ml.

For parenteral administration the invention provides ampoules or vials that include an aqueous or non-aqueous solution or emulsion. For rectal administration there are provided suppositories with hydrophilic or hydrophobic (gel) vehicles.

The methods and compositions of the invention are for preventing and/or treating congestive heart failure, cardiac hypertrophy including both atrial and ventricular hypertrophy, myocardial infarction, myocardial ischemia, myocardial ischemia and reperfusion, arrhythmias, or long-standing heart failure. In preferred embodiments, the cardiovascular disorder is congestive heart failure, and/or cardiac hypertrophy, and/or ischemia and/or arrhythmias.

The dosage and frequency of administration of the drug will depend from the age and condition of the patient, type of disorder and its severity, and will be determined according to the physician's judgment. It can be presumed that for preventive treatment of subjects susceptible to a cardiovascular disorder or disease lower doses will be needed while higher doses will be administered in acute cases.

In one embodiment of the invention, the active agent is administered alone. In other embodiments of the invention, the active agent is administered in combination with another known cardiovascular drug, either before, simultaneously or after said other cardiovascular drug.

The following examples illustrate certain features of the present invention but are not intended to limit the scope of the present invention.

### EXAMPLES

Materials and Methods

(i) Materials. Rasagiline and propargylamine were kindly donated by Teva Pharmaceutical Industries Ltd. (Petach Tikva, Israel).

(ii) Cell line H9c2. Experiments were performed on the embryonic rat heart cell line H9c2. H9c2 cells were cultured in DMEM (Biological Industries, Beit-Haemek, Israel) supplemented with 10% FCS, 50 units/ml penicillin G, 50 μg/ml streptomycin sulfate, 2 mg/ml L-glutamine and sodium pyruvate. H9c2 cells were harvested by trypsinization, washed with PBS, diluted to a concentration of 5×10⁴ cells/ml with DMEM (high glucose) and cultured at 0.5 ml/well on sterile glass cover slips in 24-well plates.

(iii) Protocols Inducing Apoptosis

(a) H₂O₂ Incubation protocol—To induce apoptosis, H9c2 cultures were exposed to H₂O₂ (0.5 μM) for 7 hours.

(b) Serum starvation—To induce apoptosis, H9c2 cultures were incubated in the culture medium containing 0% FCS for the indicated times.

(c) Activation of the Fas receptor—Fas activation was induced by incubating the cultures with recombinant human Fas Ligand (rFasL; 10 ng/ml) plus the enhancing antibody (1 μg/ml) for the indicated times, according to the manufacturer's recommendations (Alexis Biochemicals, San Diego, Calif.).

(iv) Determination of Apoptosis by DAPI. Cultures were counterstained with DAPI to visualize the nuclear morphology. Cells were scored as apoptotic, only if they exhibited unequivocal nuclear chromatin condensation and fragmentation.

In the following experiments, our major aim was to determine whether rasagiline and propargylamine can provide protection against apoptosis induced by several means in the embryonic cardiac cell line H9c2 or in neonatal rat ventricular myocytes.

### Example 1

Rasagiline Protects H9c2 Heart Cells Against Apoptosis Induced by Fas Activation

The first apoptosis-inducing protocol tested was activation of the Fas receptor with recombinant Fas Ligand (rFasL) plus the enhancing antibody (Yaniv et al., 2002).

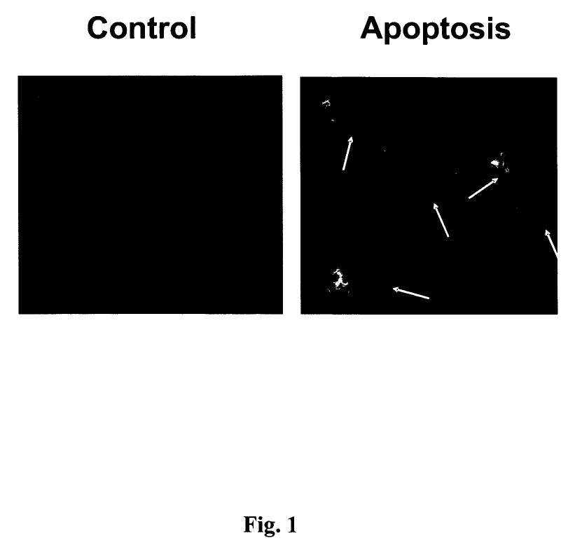

Cultures of embryonic rat heart cell line H9c2 were incubated with rFasL (10 ng/ml) and an enhancing antibody for periods of time of 9, 10 and 24 hours, and apoptosis measured thereafter. As shown in FIG. 1, Fas activation caused prominent apoptosis in H9c2 cells, as detected by the DAPI assay.

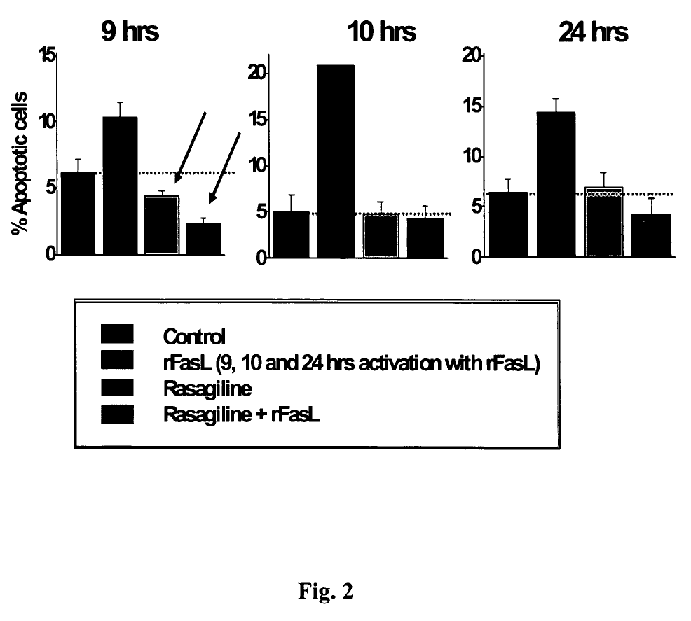

In order to determine whether rasagiline can prevent Fas-mediated apoptosis, the Fas receptor was activated for 9, 10 and 24 hours as described above. Rasagiline (10 μM) was introduced to the culture medium 16 hours before, and was present throughout the apoptosis-inducing protocol (n=3 wells). As seen in FIG. 2, the maximal apoptotic effect (˜20% apoptosis) of Fas activation was attained at 10 hours incubation with rFasL. This apoptotic effect was completely prevented by rasagiline, demonstrating that rasagiline blocks Fas-mediated apoptosis.

### Example 2

Rasagiline Protects Cultured Neonatal Rat Ventricular Myocytes Against Apoptosis Induced by Fas Activation

To test whether rasagiline can prevent the marked hypertrophy induced in cultured neonatal rat ventricular myocytes (for methods see: Yaniv et al, 2002), Fas was activated by 24 hours incubation with rFasL (10 ng/ml rFasL plus 1 μg/ml of the enhancer antibody). Hypertrophy was assessed by determining the mRNA levels (by means of RT-PCR) of the atrial natriuretic peptide (ANP), which is a most common molecular marker of hypertrophy. Rasagiline (10 μM/ml )was added to the culture 1 hour before Fas activation and remained in the medium throughout the 24 hours exposure to rFasL. In these preliminary experiments we have found that rasagiline prevented Fas-mediated hypertrophy, a finding which may have an important clinical significance.

### Example 3

Rasagiline Protects H9c2 Heart Cells Against Apoptosis Induced by Serum Starvation

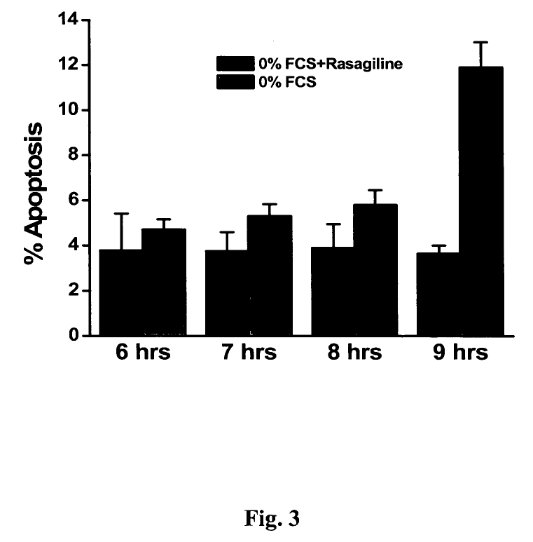

The next apoptosis-inducing stimulus tested was serum starvation (24 hrs, 0% serum in the culture medium). To induce apoptosis, H9c2 cells were incubated in the culture medium containing 0% FCS for 6, 7, 8 or 9 hours. Rasagiline was introduced to the culture medium 2 hours before inducing serum starvation and was present throughout the apoptosis-inducing protocol (n=3 wells). As seen in FIG. 3, the most effective protocol was 9 hrs serum starvation, which caused 12% apoptosis. This effect was completely prevented by rasagiline (10 μM).

### Example 4

Rasagiline Protects H9c2 Heart Cells Against Apoptosis Induced by Serum Starvation but not H₂O₂-Induced Apoptosis

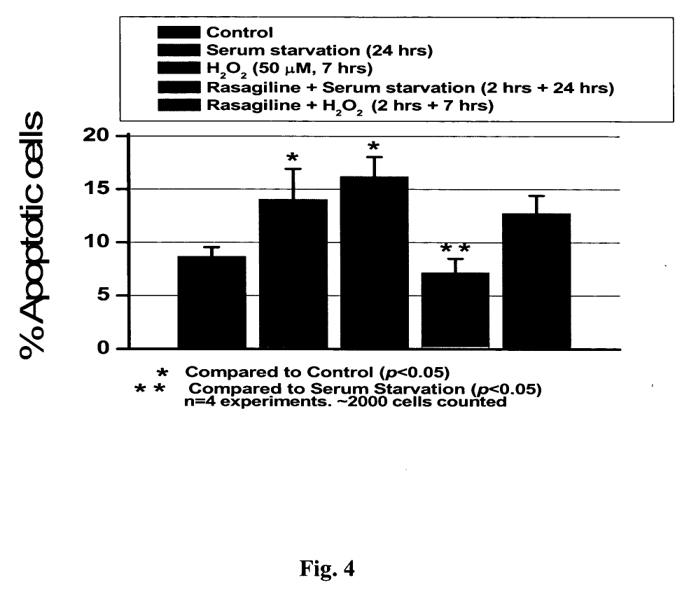

In another experiment, we repeated the serum starvation protocol, and also tested in the same cultures whether rasagiline can protect against H₂O₂-induced apoptosis. Rasagiline was introduced to the culture medium 2 hours before inducing serum starvation or adding H₂O₂, and was present throughout the apoptosis-inducing protocol (n=4 experiments; about 2000 cells counted). As clearly shown in FIG. 4, rasagiline prevented the apoptosis induced by serum starvation (green bar), but not by H₂O₂ (gray bar).

In summary, rasagiline prevented apoptosis induced by Fas activation (as well as the hypertrophy) and by serum starvation, which may have an immense clinical importance for the prevention and treatment of major cardiac pathologies.

### Example 5

Propargylamine Blocks Hypertrophy Induced by Activation of the Fas Receptor in Cultures of Neonatal Rat Ventricular Myocytes

To test whether propargylamine can prevent the marked hypertrophy induced in cultures of neonatal rat ventricular myocytes (for methods, see Yaniv et al., 2002), Fas was activated for 24 hours with 10 ng/ml rFasL plus 1 μg/ml enhancer antibody (Yaniv et al., 2002). Hypertrophy was assessed by determining the mRNA levels (by means of RT-PCR) of ANP, which is a most common molecular marker of hypertrophy. Ten μM of propargylamine were added to the appropriate culture 60 minutes before Fas activation.

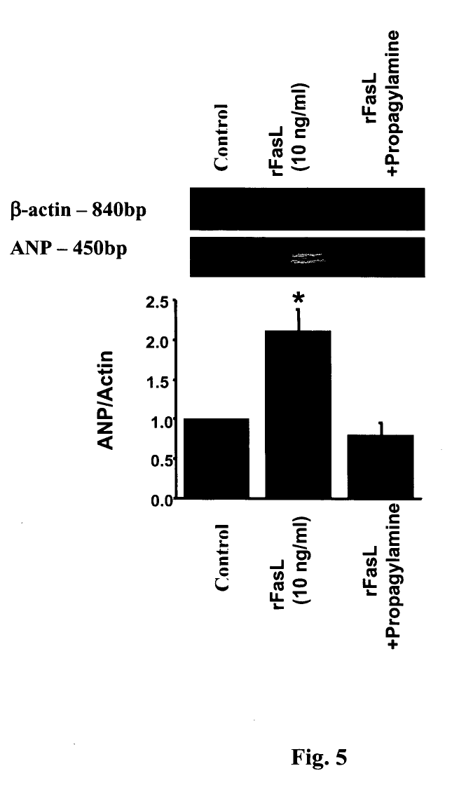

As shown in FIG. 5, ANP mRNA elevation induced by incubation with rFasL for 24 h was blocked by 10 μM of propargylamine. n=3 experiments, *P<0.05 versus control. We conclude that propargylamine protects ventricular myocytes against hypertrophy caused by activation of the Fas receptor and this finding can have an important clinical significance.

### REFERENCES

Berke G. 1997. The Fas-based mechanism of lymphotoxicity. Human Immunol 54:1-7.

Binah 0. 2000. Immune effector mechanisms in myocardial pathologies. Int J Mol Med 6:3-16.

Durden D A, Dyck L E, Davis B A, Liu Y D, Boulton A A. 2000. Metabolism and pharmacokinetics, in the rat, of (R)-N-(2-Heptyl)Methyl-propargylamine (R-2HMP), a new potent monoamine oxidase inhibitor and antiapoptotic agent. Drug Metab Dispos. 28(2):147-54.

Fliss H, Gattinger D. 1996. Apoptosis in ischemic and reperfused rat myocardium. Circ Res 79:949-956.

Haunstetter A, Izumo S. 1998. Apoptosis: basic mechanisms and implications for cardiovascular disease. Circ Res 82:1111-1129.

Hershkowitz A, Wolfgram L J, Rose N R et al. 1987. Coxsackievirus B3 murine myocarditis: a pathologic spectrum of myocarditis in genetically defined inbred strains. J Am Coll Cardiol 9:1311-1319.

Jeremias I, Kupatt C, Villalba-Martin et al. 2000. Involvement of CD95/Apo-1/Fas in cell death after myocardial ischemia. Circulation 102:915-920.

Tatton, et al., 1993. Selegiline Can Mediate Neuronal Rescue Rather than Neuronal Protection. Movement Disorders 8 (Supp. 1):S20-S30.

Tatton, et al. 1991. Rescue of Dying Neurons,″ J. Neurosci. Res. 30:666-672.

Yamaguchi s, Yamaoka M, Okuyama M et al. 1998. Elevated circulation levels and cardiac secretion of soluble Fas ligand in patients with congestive heart failure. Am J Cardiol 83:1500-1503.

Yaniv G, Shilkrut M, Lotan R, Larish S, Berke G, Binah 0. 2002. “Hypoxia predisposes neonatal rat ventricular myocytes to apoptosis induced by activation of the Fas (CD95/Apo-1) receptor: Fas activation and apoptosis in hypoxic myocytes”. Cardiovasc Res 54:611-623.

Yaoita H, Ogawa K, Macehara K, Maruyama Y. 1998. Attenuation of ischemia/reperfusion injury in rats by a caspase inhibitor. Circulation 97:276-281.

Zimmermann K, Waldmeier P C, Tatton W G. 1999. Dibenzoxepines as treatments for neurodegenerative diseases. Pure Appl Chem 71(11):2039-2046.

1. A method for treatment of a subject susceptible to or suffering from a cardiovascular disorder or disease which comprises administering to the subject an amount of an active agent selected from the group consisting of propargylamine, a propargylamine derivative, and a pharmaceutically acceptable salt thereof, effective to treat the subject.

2. A method according to claim 1 for preventing and/or treating congestive heart failure, cardiac hypertrophy including atrial and ventricular hypertrophy, myocardial infarction, myocardial ischemia, myocardial ischemia and reperfusion, or arrhythmias.

3. A method according to claim 1 wherein said active agent is selected from the group consisting of N-propargyl-1-aminoindan, an enantiomer thereof, an analog thereof, and a pharmaceutically acceptable salt of the aforesaid,

4. A method according to claim 3 wherein said active agent is racemic N-propargyl-1-aminoindan.

5. A method according to claim 3 wherein said active agent is the enantiomer R(+)-N-propargyl-1-aminoindan.

6. A method according to claim 3 wherein said active agent is the enantiomer is S-(−)-N-propargyl-1-aminoindan.

7. A method according to claim 3 wherein said active agent is a pharmaceutically acceptable salt of R(+)-N-propargyl-1-aminoindan.

8. A method according to claim 7 wherein said pharmaceutically acceptable salt is selected from the group consisting of the mesylate salt; the esylate salt; the sulfate salt; and the hydrochloride salt of R(+)-N-propargyl-1-aminoindan.

9. A method according to claim 3 wherein said analog of N-propargyl-1-aminoindan is selected from the group consisting of 4-fluoro-N-propargyl- 1-aminoindan, 5-fluoro-N-propargyl-1-aminoindan, 6-fluoro-N-propargyl- 1-amino-indan, an enantiomer thereof and pharmaceutically acceptable addition salts thereof.

10. A method according to claim 3 wherein said analog of N-propargyl-1-aminoindan is selected from the group consisting of: (rac)-3-(N-methyl,N-propyl-carbamyloxy)-α-methyl-N′-propargyl phenethylamine HCl; (rac)-3-(N,N-dimethyl-carbamyloxy)-α-methyl-N′-methyl, N′-propargyl phenethylamine HCl; (rac)-3-(N-methyl,N-hexyl-carbamyloxy)-α-methyl-N′-methyl, N′-propargyl phenethylamine mesylate; (rac)-3-(N-methyl,N-cyclohexyl-carbamyloxy)-α-methyl-N′-methyl,N′-propargyl phenethylamine HCl; and (S)-3-(N-methyl, N-hexyl-carbamyloxy)-α-methyl-N′-methyl,N′-propargyl phenethylamine ethanesulfonate.

11. A method according to claim 3 wherein said analog of N-propargyl-1-aminoindan is selected from the group consisting of: (rac) 6-(N-methyl, N-ethyl-carbamyloxy)-N′-propargyl-1-aminoindan HCl; (rac) 6-(N,N-dimethyl, carbamyloxy)-N′-methyl-N′-propargyl-1-aminoindan HCl; (rac) 6-(N-methyl, N-ethyl-carbamyloxy-N′-propargyl-1-aminotetralin HCI; (rac) 6-(N,N-dimethyl-thiocarbamyloxy)-1-aminoindan HCl; (rac) 6-(N-propyl-carbamyloxy-N′-propargyl-1-aminoindan HCl; (rac) 5-chloro-6-(N-methyl, N-propyl-carbamyloxy)-N′-propargyl-1-aminoindan HCl; (S)-6-(N-methyl), N-propyl-carbamyloxy)-N′-propargyl-1-aminoindan HCl; and (R)-6-(N-methyl, N-ethyl-carbamyloxy)-N′-propargyl-1-aminoindan hemi-(L)-tartrate. 6 and 6-(N-methyl, N-ethyl-carbamyloxy)-N′-methyl,N′-propargyl-1-aminoindan.

12. A method according to claim 1 wherein said active agent is an aliphatic propargylamine.

13. A method according to claim 12 wherein said aliphatic propargylamine is selected from the group consisting of the compounds N-(1-heptyl)propargylamine or a propargylamine; N-(1-octyl) propargylamine; N-(1-nonyl) propargylamine; N-(1-decyl)propargyl-amine; N-(1-undecyl)propargylamine: N-(1-dodecyl) propargylamine; R-N-(2-butyl)propargylamine; R-N-(2-pentyl) propargylamine; R-N-(2-hexyl) propargylamine; R-N-(2-heptyl)propargylamine; R-N-(2-octyl) propargylamine; R-N-(2-nonyl)propargylamine; R-N-(2-decyl) propargylamine, R-N-(2-undecyl) propargylamine; R-N-(2-dodecyl)propargylamine: N-(1-butyl)-N-methylpropargyl-amine; N-(2-butyl)-N-methylpropargylamine; N-(2-pentyl)-N-methylpropargyl-amine; N-(1-pentyl)-N-methylpropargylamine; N-(2-hexyl)-N-methylpropargyl-amine; N-(2-heptyl)-N-methylpropargylamine; N-(2-decyl)-N-methylpropargyl-amine; N-(2-dodecyl)-N-methylpropargylamine; R(−)-N-(2-butyl)-N-methyl-propargylamine; or a pharmaceutically acceptable salt thereof.

14. A method according to claim 1 wherein said active agent is selected from the group consisting of selegiline, desmethylselegiline, pargyline and chlorgyline.

15. A method according to claim 1 wherein said active agent is the compound (N-methyl-N-propargyl-10-aminomethyl-dibenzo[b,f]oxepin.

16. A method according to claim 1 wherein said active agent is propargylamine or a pharmaceutically acceptable salt thereof.

17. A method according to claim 1 for protecting ventricular muscle from apoptosis, wherein said cardiovascular disorder or disease is ischemia/reperfusion injury, myocardial infarction, and long-standing heart failure.

18. A method according to claim 17, wherein said apoptosis is Fas-mediated apoptosis.

19. An article of manufacture comprising packaging material and a pharmaceutical composition contained within the packaging material, said pharmaceutical composition comprising an active agent selected from the group consisting of propargylamine, a propargylamine derivative and a pharmaceutically acceptable salt thereof, and said packaging material includes a label that indicates that said agent is therapeutically effective for treating a cardiovascular disease or disorder.
